(19) United States
(12) Patent Application Publication
(10) Pub. No.: US 2019/0000889 A1
(43) Pub. Date: Jan. 3, 2019

(54) METHODS FOR TREATING DIABETES

(71) Applicant: CITY OF HOPE, Duarte, CA (US)

(72) Inventor: Defu ZENG, Arcadia, CA (US)

(21) Appl. No.: 16/067,605

(22) PCT Filed: Nov. 10, 2016
(86) PCT No.: PCT/US2016/061403

§ 371 Date: Jun. 30, 2018

Related U.S. Application Data
(60) Provisional application No. 62/274,083, filed on Dec. 31, 2015.

(51) Int. Cl. A61K 35/39; A61P 3/10; A61K 38/22; A61K 31/675; A61K 31/7056; A61K 39/395; A61K 35/28

(52) U.S. Cl. CPC A61K 35/39; A61P 3/10; A61K 38/2207; A61K 35/28; A61K 31/7056; A61K 39/3955; A61K 31/675

(57) ABSTRACT

Disclosed herein are methods for treating type 1 diabetes by reversing autoimmunity and replenishing beta cells. More specifically, type 1 diabetes is treated by administering exogenous Sox9+ cells and a low dose of gastrin and epidermal growth factor (GE) under hyperglycemia or medium hyperglycemia condition during or after induction of mixed chimerism in a subject.

### PRIORITY CLAIM

This application claims priority to U.S. Provisional Application No. 62/274,083, filed Dec. 31, 2015, which is incorporated by reference herein in its entirety, including drawings.

### BACKGROUND

Autoimmune type 1 diabetes (T1D) results from autoimmune attack on insulin-secreting β cells and subsequent insulin deficiency (1). Cure of T1D requires both reversal of autoimmunity and resupply of insulin-secreting β cells by islet transplantation or augmentation of endogenous β cell regeneration (2). Due to the lack of donors for islet transplantation and islet grafts lasting only for about 3 years (3), augmentation of endogenous β cell regeneration would be the more favorable approach. It was previously reported that combination therapy of induction of mixed chimerism and administration of gastrin and epidermal growth factor (GE) not only reversed autoimmunity but also augmented β cell neogenesis and replication and subsequently cured late-stage T1D in autoimmune NOD mice (4). However, the origin of neogenesis remains unknown.

It has been proposed that β cell neogenesis in adult mice can derive from pancreatic ductal cells (5, 6), cells in the islet (7, 8), transdifferentiation from glucagon-producing α cells (9, 10) or from acinar cells (11-13). Although it has been consistently reported that pancreatic ductal epithelial cells give rise to insulin-producing β cells during embryonic development (14-17), whether the pancreatic ductal progenitors can give rise to insulin-producing β cells in neonates and adult mice remains controversial. Using a Cre-based lineage tracing with a human carbonic anhydrase II (CAII) promoter, Inada et al reported that pancreatic ductal cells were able to give rise to insulin-producing β cells in neonates and in PDL-treated adult mice (5). Using Cre-based lineage tracing model with a Ngn3 promoter, Xu et al also found that cells in the pancreatic ductal lining could give rise to β cells in PDL-treated adult mice (6). On the other hand, using a lineage-tracing model with a Hnf1β promoter, Solar et al found that pancreatic ductal cells did not give rise to β cells in neonates, PDL-treated adult mice, or Alloxan-induced diabetic adult mice treated for one week with GE (14). Using a lineage-tracing model with a Sox9 promoter, Kopp et al also showed that Sox9⁺ ductal cells did not give rise to β cells postnatally after β cell ablation or after PDL (17, 18). Similarly, Furuyama et al reported that Sox9⁺ pancreatic ductal cells were not able to give rise to β cells in PDL-treated adult mice or STZ-induced diabetic mice (16). These reports suggest that PDL injury with normal glycemia, hyperglycemia alone, or hyperglycemia plus short-term (1 week) administration of GE is not able to augment pancreatic Sox9⁺ ductal cells differentiation into insulin-producing β cells in adult mice. Actually, how hyperglycemia regulates progenitor differentiation into β cells remains largely unknown, although it has been reported that hyperglycemia is toxic to β cells (19, 20).

Several reports have shown that a subpopulation of cells in the pancreatic ducts of adult mice is clonogenic and can give rise to insulin-producing β cells in various in vitro culture systems (21, 22). One recent study, which uses in vitro semisolid medium culture, has shown that Sox9⁺CD133⁺ pancreatic ductal cells from adult mice are able to give rise to three cell lineages, including insulin-producing β cells, ductal epithelial cells, and acinar cells (23, 24). Although treatment with GE was reported to induce β cell neogenesis from adult pancreatic ductal cells in vitro (25), it was not successfully applied clinically.

There remains a need to develop an effective therapy for type 1 diabetes. This disclosure provides methods for treating type 1 diabetes to satisfy the need.

### SUMMARY

In one aspect, the disclosure provided herein relates to methods for treating type 1 diabetes in a subject. The methods entail a two-pronged treatment, including reversing autoimmunity and replenishing pancreatic β cells in a subject. In some embodiments, autoimmunity is reversed, for example, by inducing stable mixed chimerism in the subject. In some embodiments, pancreatic β cells are replenished by administering to the subject an effective amount of Sox9⁺ cells; and an effective amount of gastrin and epidermal growth factor (GE). In such embodiments, the Sox9⁺ cells act as progenitor cells for pancreatic β cells. Preferably, GE is administered at a low dose and/or for an extended period of time. Preferably, GE is administered to the subject under hyperglycemia or medium hyperglycemia condition.

In some embodiments, the methods for treating type 1 diabetes in a subject disclosed herein include administering to the subject low-doses of cyclophosphamide (CY), pentostatin (PT), and anti-thymocyte globulin (ATG); transplanting in the subject a therapeutically effective amount of donor bone marrow cells; administering to the subject an effective amount of Sox9⁺ cells; and administering to the subject an effective amount of gastrin and epidermal growth factor (GE). In certain embodiments, two or more steps are carried out simultaneously.

In a related aspect, the disclosure relates to a composition for treating type 1 diabetes in a subject. The composition includes one or more of the agents disclosed herein, one or more populations of the cells disclosed herein, or one or more combinations of agents and populations of the cells disclosed herein. In some embodiments, the composition includes Sox9⁺ cells and bone marrow stem cells. In some embodiments, the composition further includes one or more of cyclophosphamide (CY), pentostatin (PT), anti-thymocyte globulin (ATG), gastrin, and epidermal growth factor. In yet other embodiments, the composition further includes one or more populations of conditioning donor cells selected from donor CD4⁺ T-depleted spleen cells, donor CD8⁺ T cells, and donor Granulocyte colony-stimulating factor (G-CSF)-mobilized peripheral blood mononuclear cells.

### BRIEF DESCRIPTION OF THE DRAWINGS

This application contains at least one drawing executed in color. Copies of this application with color drawing(s) will be provided by the Office upon request and payment of the necessary fees.

FIGS. 1A-1K show long-term administration of low-dose GE reverses diabetes in C57BL/6 mice with medium hyperglycemia. Adult female C57BL/6 mice were induced to develop diabetes by I.P. injection of one dose of Alloxan (70 mg/kg). 28 days after injection of Alloxan, diabetic mice with medium (300-450 mg/dL) and high (>500 mg/dL) hyperglycemia were selected for experiments and were treated with Gastrin (3 μg/kg) plus EGF (1 μg/kg) (GE) for 56 days. Thereafter, the mice were monitored for another 56 days. The mice were monitored for body weight and blood glucose twice a week for up to 140 days. Before ending the experiments, mice were measured with an intraperitoneal glucose tolerance test (IPGTT) and for insulin secretion. After ending the experiments, pancreases were harvested and stained for insulin to measure β cell surface. FIG. 1A shows experimental scheme. FIGS. 1B-1F show that diabetic mice with medium hyperglycemia were treated with Gastrin plus EGF, and then monitored for blood glucose for 16 weeks. FIG. 1B shows blood glucose levels of diabetic C57BL/6 mice treated with GE or PBS control. Mice with “Reversal of diabetes” had lasting stable blood glucose levels that was close to 200 mg/dL. There were 12 mice in each group combined from three experiments. FIG. 1C shows the percentage of body weight change (Mean±SEM, n=12). FIG. 1D shows IPGTT blood glucose (n=6). FIG. 1E shows serum insulin levels before and 10 minutes after glucose injection during IPGTT (Mean±SEM, n=6). FIG. 1F shows the percentage of β cell surface (Mean±SEM, n=4). FIGS. 1G-1K show that diabetic mice with high hyperglycemia were treated with Gastrin plus EGF and monitored as described above. FIG. 1G shows blood glucose levels of diabetic C57BL/6 mice treated with GE or PBS control. There were 12 mice in each group combined from three experiments. FIG. 1H shows the percentage of bodyweight change (Mean±SEM, n=12). FIG. 1I shows IPGTT blood glucose (n=6). FIG. 1J shows serum insulin levels before and 10 minutes after glucose injection during IPGTT (Mean±SEM, n=6). FIG. 1K shows the percentage of β cell surface (Mean±SEM, n=4) (* P<0.05; **P<0.01; ***P<0.001).

FIGS. 2A-2E show that long-term administration of low-dose GE augments both β cell neogenesis and replication in diabetic mice with medium but not high hyperglycemia. 8 weeks old female Ins1^{CreERT}R26^{mT/mG} mice were first given I.P. injection of Tamoxifen™ every two days over a 2-week period to induce EGFP expression and label the pre-existing β cells. Thereafter, mice were induced to develop diabetes with Alloxan, and mice with medium or high hyperglycemia were treated with GE or PBS control. At the end of the experiments, the pancreases were harvested and stained for insulin and EGFP, and merged staining was also shown. Nuclei were labeled by DAPI. The percentage of EGFP⁺Ins⁺ cells amongst total Insulin⁺ cells was calculated. Normal non-diabetic mice were used as a control for checking EGFP labeling efficiency. FIG. 2A shows experimental scheme. FIG. 2B shows that the islets in mice with normal or medium hyperglycemia after PBS or GE treatment were stained for Insulin (red), EGFP (green) or merged colors (original magnification 400×). FIG. 2C shows the percentage of Ins/EGFP⁺Ins⁺ cells among Insulin⁺ cells in islets of mice in 2B (Mean±SEM, n=4). FIG. 2D shows representative staining pattern of islets in mice with high hyperglycemia after PBS or GE treatment (original magnification 400×). FIG. 2E shows the percentage of Ins/EGFP⁺Ins⁺ cells among Insulin⁺ cells in the islets of mice in D (Mean±SEM, n=4).

FIGS. 3A-3F show that long-term administration of low-dose GE augments Sox9⁺ ductal cell differentiation into β cells in diabetic mice with medium hyperglycemia. 8 weeks old female Sox9^{CreERT2} R26^{mT/mG} mice were first injected with Tamoxifen™ to label the Sox9/EGFP⁺ cells, then the mice were induced to develop diabetes and treated with PBS or GE and monitored as described in FIG. 2A. At the end of experiments, pancreases were stained for Insulin and EGFP, and merged staining was also shown. FIG. 3A shows representative pattern of EGFP labeling Sox9⁺ pancreatic ductal cells in the normal glycemia control mice (original magnification 400×). FIG. 3B shows quantification of Sox9⁺ EGFP⁺ cells relative to the total number of Sox9⁺ cells in the duct in A (Mean±SEM, n=4). FIG. 3C shows representative staining pattern of islets in mice with normal or medium hyperglycemia after PBS or GE treatment (original magnification 400×). FIG. 3D shows the percentage of Sox9/EGFP⁺Ins⁺ cells among total Insulin⁺ cells in islets of mice in C (Mean±SEM, n=4). FIG. 3E shows representative staining pattern of islets in mice with high hyperglycemia after PBS or GE treatment (original magnification 400×). FIG. 3F shows the percentage of Sox9/EGFP⁺Ins⁺ cells among total Insulin⁺ cells in islets of mice in E (Mean±SEM, n=4).

FIGS. 4A-4E show long-term administration of low-dose GE increases insulin^{lo} cells among Sox9/EGFP⁺CD133⁺ cells although it does not increase the percentage of Sox9/EGFP⁺CD133⁺ in diabetic mice with medium hyperglycemia. After treated by TM and Alloxan as described in FIG. 2A, the pancreatic tissues from normal and diabetic Sox9^{CreERT2} R26^{mT/mG} mice with medium hyperglycemia after 56 days treatment with PBS or GE were dissociated into single cells. Sox9/EGFP⁺CD133⁺, Insulin⁺, Insulin⁺Glucagon⁺ cell population were analyzed by flow cytometry. FIGS. 4A and 4B show the percentage of Sox9/EGFP⁺CD133⁺ among total pancreatic mononuclear cells (Mean±SEM, n=4). FIGS. 4C and 4D show the percentage of Insulin⁺ and Insulin⁺Glucagon⁺ cells after gating on Sox9/EGFP⁺CD133⁺ cell population. FIG. 4E shows that pancreatic tissues were stained for Insulin, EGFP, and Pdx1 or NKX6.1. One representative staining pattern of islets in mice is shown from 4 replicate experiments. Triple-positive cells are shown in the box. Scale bar=30 μm.

FIGS. 5A and 5B show that long-term administration of low-dose GE induces the presence of Sox9/EGFP⁺CD133⁺Ins⁺ or Sox9/EGFP⁺Ins⁺Glu⁺ triple-positive cells in the islets of diabetic mice with medium hyperglycemia. After treated by TM and Alloxan as described in FIG. 2A, the pancreatic tissues from diabetic Sox9^{CreERT2}R26^{mT/mG} mice withmedium hyperglycemia after 56-day-treatment with GE were stained for Insullin, EGFP and CD133 or Glucagon. FIG. 5A shows cells co-stained with Sox9/EGFP⁺CD133⁺Ins⁺ in islet in the box. Scale bar=20 μm.

FIG. 5B shows cells co-stained with Sox9/EGFP⁺Ins⁺Glu⁺ in immature islet in the box in upper row and cells co-stained with Sox9/EGFP⁺Ins⁺Glu⁻ in mature islet in the box in bottom row. Scale bar=40 μm.

FIGS. 6A-6D show that medium hyperglycemia is required for inducing Sox9⁺ ductal cells differentiation into β cells. After treated by TM and Alloxan as described in FIG. 2A, Sox9^{CreERT2} R26^{mT/mG} mice with high hyperglycemia were chosen for long-term administration of low-dose GE. During GE treatment, insulin pellets were implanted to completely control hyperglycemia to normal level (<200 mg/dL) or partially control to medium level (200-450 mg/dL). At the ending of GE treatment, pancreatic tissues were stained for Insulin and EGFP, and merged staining was also shown. FIG. 6A shows experimental scheme. FIG. 6B shows blood glucose levels from GE+ Complete control and GE+ Partial control groups (n=6). FIG. 6C shows representative staining pattern of islets in mice from GE+ Non-diabetic, GE+ Complete control and GE+ Partial control groups (original magnification 400×). FIG. 6D shows quantification of Sox9/EGFP⁺Ins⁺ relative to the total number of Insulin⁺ cells in islets of mice in C (Mean±SEM, n=4).

FIGS. 7A-7F show short-term administration of high-dose GE does not augment β cell neogenesis from Sox9⁺ ductal cells in diabetic mice with medium hyperglycemia. After treated by TM and Alloxan as described in FIG. 2A, Sox9^{CreERT2}R26^{mT/mG} mice with medium hyperglycemia were chosen for short-term administration of high-dose GE. Alzet osmotic mimipumps were intraperitoneally implanted for 7 days, followed by 21 days of monitoring. The pumps contained Gastrin (release rate: 3 μg/kg body weight per hour) and EGF (release rate: 10 μg/kg body weight per hour), and release their content for approximately 7 days. At the ending of high-dose GE treatment, pancreatic tissues were stained for Insulin and EGFP, and merged staining was also shown. FIG. 7A shows experimental scheme. FIG. 7B shows blood glucose levels from PBS-pump (n=6) and GE-pump (n=10) groups. FIG. 7C shows representative staining pattern of islets in mice from PBS-pump and GE-pump groups (original magnification 400×). FIG. 7D shows quantification of Sox9/EGFP⁺Ins⁺ cells relative to the total number of Insulin⁺ cells in islets of mice in C (Mean±SEM, n=4). FIG. 7E shows that to determine short-term administration of high-dose GE induced β cell replication or not, Ins1^{CreERT}R26^{mT/mG} mice with medium hyperglycemia were treated with a PBS or GE pump described as FIG. 7A. Representative staining pattern of islets in mice from PBS-pump and GE-pump groups is shown (original magnification 400×). FIG. 7F shows quantification of Ins/EGFP⁺Ins⁺ cells relative to the total number of Insulin⁺ cells in the islets of mice in E (Mean±SEM, n=4).

FIGS. 8A-8D show that long-term administration of low-dose GE increases Sox9/EGFP⁺Insulin⁺ cells on the wall of pancreatic ducts. Sox9^{CreERT2}R26^{mT/mG} mice were induced to label pancreatic ductal epithelial cells with TM and then induced to diabetes with Alloxan. Normal non-diabetic mice and diabetic mice with medium or high hyperglycemia were treated with control PBS or GE for 8 weeks, followed by 8 weeks of monitoring as described in FIG. 2A. At the end of the experiments, pancreatic tissues were stained for Insulin, EGFP, and Glucagon, and merged staining was also shown. FIGS. 8A-8C show representative staining pattern of pancreatic ducts were shown from mice with normal glycemia, medium hyperglycemia, and high hyperglycemia after long-term administration of PBS or low-dose GE (original magnification 400×). FIG. 8D shows number of Sox9/EGFP⁺Ins⁺ cells on the wall of pancreatic ducts per tissue section in A-C (Mean±SEM, n=4).

FIGS. 9A and 9B show kinetic analysis of small islets (5 cells) in Sox9^{CreERT2}R26^{mT/mG} mice and Ins1^{CreERT}R26^{mT/mG} mice early after induction of diabetes. TM-treated Sox9^{CreERT2}R26^{mT/mG} mice and Ins1^{CreERT}R26^{mT/mG} mice were given injection of Alloxan as described above (FIG. 2A). The mice with hyperglycemia (blood glucose >400 mg/dL) on days 2, 4, 8 after Alloxan injection were used for kinetic analysis of small islets (5 cells). Mice before injection (day 0) were used as control. The pancreatic tissues were stained for insulin and EGFP. The numbers of small islets (5 cells) per section was calculated. FIG. 9A shows Sox9/EGFP⁻Ins⁺ and Sox9/EGFP⁺Ins⁺ small islets per section in Sox9^{CreERT2}R26^{mT/mG} mice (Mean±SEM, n=4). FIG. 9B shows Ins/EGFP⁺Ins⁺ and Ins/EGFP⁻Ins⁺ small islets in Ins1^{CreERT}R26^{mT/mG} mice (Mean±SEM, n=4).

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

Disclosed herein are methods for treating diabetes, particularly autoimmune type 1 diabetes. The methods entail a two-pronged treatment: (1) reversing autoimmunity, for example, by inducing mixed chimerism; and (2) replenishing 13 cells in a subject in need or suffering from autoimmune type 1 diabetes.

According to the embodiments described herein, the methods include administrating a population of Sox9⁺ cells from a donor or a recipient, and administering a low dose of gastrin and epidermal growth factor (together, “GE”) for an extended period of time, to a subject in need under a hyperglycemia (>300 mg/dL) or medium hyperglycemia (300-450 mg/dL) condition to induce differentiation of Sox9⁺ cells into pancreatic β cells. In such embodiments, the Sox9⁺ cells act as progenitor cells for pancreatic β cells.

In some embodiments, a gastrin receptor agonist, in lieu of gastrin, can be used in combination of epidermal growth factor to activate gastric receptors and induce differentiation of Sox9⁺ cells into β cells in the methods and compositions disclosed herein. The term “GE” as used herein may be inclusive of the use of gastrin or a gastrin receptor agonist with epidermal growth factor Exemplary gastrin receptor agonists include, but are not limited to, cholecystokinin, CCK-4, BBL-454 and any functional fragment thereof.

In addition to providing Sox9⁺ cells and inducing their differentiation into 13 cells, the methods disclosed herein further comprise reversing autoimmunity before or during administrating Sox9⁺ cells to the subject. In some embodiments, the Sox9⁺ cells are administered to the subject after reversal of autoimmunity. Autoimmunity of the subject can be reversed by inducing mixed chimerism.

The inventors have unexpectedly discovered that synergistic effects can be achieved to augment the differentiation of Sox9⁺ progenitors into β cells when exogenous Sox9⁺ cells from either a donor or a recipient are administered and a low dose of GE is administered for an extended period of time under hyperglycemia condition, thereby to treat type 1 diabetic patients after reversal of autoimmunity. The therapeutic effects are even more enhanced under medium hyperglycemia condition.

### Definitions

The term “recipient” or “host” as used herein refers to a subject receiving transplanted or grafted tissue or cells, or a treatment or a therapy. These terms may refer to, for example, a subject receiving an administration of Sox9⁺ cells, G-CSF mobilized peripheral blood mononuclear cells, donor bone marrow, donor T cells, or a tissue graft. The transplanted tissue may be derived from a syngeneic or allogeneic donor. The recipient, donor, host, patient, or subject in this disclosure can be an animal, a mammal, or a human. In one embodiment, the recipient or host is a subject that has Type I diabetes that is treated with insulin.

The term “donor” as used herein refers to a subject from whom tissue or cells such as Sox9⁺ cells are obtained to be transplanted or grafted into a recipient or host. For example, a donor may be a subject from whom bone marrow, Sox9⁺ cells, T cells, or other tissue to be administered to a recipient or host is derived. The donor or subject can be an animal, a mammal, or a human. In certain embodiments, the donor for inducing mixed chimerism may be an MHC- or HLA-matched donor, meaning the donor shares the same MHC- or HLA with the recipient. In certain embodiments, the donor may be MHC- or HLA-mismatched to the recipient.

The term “hyperglycemia” generally means that the blood glucose level of a subject is higher than 11.1 mmol/L (200 mg/dL). However, diabetic symptoms may not become noticeable until the blood glucose reaches a higher level, such as 15-20 mmol/L (about 250-300 mg/dL). A subject having a consistent range of blood glucose between about 5.6 mmol/L and about 7 mmol/L (100-126 mg/dL) is considered hyperglycemic, while above 7 mmol/L (126 mg/dL) is considered to have diabetes according to the American Diabetes Association guidelines. In the context of this disclosure, the treatment to replace or replenish β cells achieves an improved therapeutic effect under a hyperglycemia (a blood glucose level >300 mg/dL) or medium hyperglycemia (a blood glucose level of 300-450 mg/dL) condition.

The term “chimerism” as used herein refers to a state in which one or more cells from a donor are present and functioning in a recipient or host. Recipient tissue exhibiting “chimerism” may contain donor cells only (complete chimerism), or it may contain both donor and host cells (mixed chimerism). “Chimerism” as used herein may refer to either transient or stable chimerism. In some embodiments, the mixed chimerism may be MHC- or HLA-matched mixed chimerism. In certain embodiments, the mixed chimerism may be MHC- or HLA-mismatched mixed chimerism.

The terms “treat,” “treating,” or “treatment” as used herein with regards to a condition refers to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. In some embodiments, treating a condition means that the condition is cured without recurrence.

The phrase “a therapeutically effective amount” or “an effective amount” as used herein refers to an amount of an agent, population of cells, or composition that produces a desired therapeutic effect. For example, a therapeutically effective amount of donor BM cells or donor CD4⁺ T-depleted spleen cells may refer to that amount that generates chimerism in a recipient. In another example, a therapeutically effective amount of Sox9⁺ cells may refer to an amount of Sox9⁺ cells producing sufficient amount of β cells. The precise therapeutically effective amount is an amount of the agent, population of cells, or composition that will yield the most effective results in terms of efficacy in a given subject. This amount will vary depending upon a variety of factors, including but not limited to the characteristics of the therapeutic agent, population of cells, or composition (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of an agent, population of cells, or composition and adjusting the dosage accordingly. For additional guidance, see Remington: The Science and Practice of Pharmacy (Gennaro ed. 20th edition, Williams & Wilkins PA, USA) (2000).

A “pharmaceutically acceptable carrier” as used herein refers to a pharmaceutically acceptable material, composition, or vehicle that is involved in carrying or transporting an agent or cell of interest from one tissue, organ, or portion of the body to another tissue, organ, or portion of the body. Such a carrier may comprise, for example, a liquid, solid, or semi-solid filler, solvent, surfactant, diluent, excipient, adjuvant, binder, buffer, dissolution aid, solvent, encapsulating material, sequestering agent, dispersing agent, preservative, lubricant, disintegrant, thickener, emulsifier, antimicrobial agent, antioxidant, stabilizing agent, coloring agent, or some combination thereof. Each component of the carrier is “pharmaceutically acceptable” in that it must be compatible with the other ingredients of the composition and must be suitable for contact with any tissue, organ, or portion of the body that it may encounter, meaning that it must not carry a risk of toxicity, irritation, allergic response, immunogenicity, or any other complication that excessively outweighs its therapeutic benefits.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) natural polymers such as gelatin, collagen, fibrin, fibrinogen, laminin, decorin, hyaluronan, alginate and chitosan; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as trimethylene carbonate, ethyl oleate and ethyl laurate; (13) agar; (14) buffering agents, such as magnesium hydroxide and aluminum hydroxide; (15) alginic acid (or alginate); (16) pyrogen-free water; (17) isotonic saline; (18) Ringer's solution; (19) alcohol, such as ethyl alcohol and propane alcohol; (20) phosphate buffer solutions; (21) thermoplastics, such as polylactic acid, polyglycolic acid, (22) polyesters, such as polycaprolactone; (23) self-assembling peptides; and (24) other non-toxic compatible substances employed in pharmaceutical formulations such as acetone.

The term “simultaneously” as used herein with regards to administration of two or more agents means that the agents are administered at the same or nearly the same time. For example, two or more agents are considered to be administered “simultaneously” if they are administered via a single combined administration, two or more administrations occurring at the same time, or two or more administrations occurring in succession without extended intervals in between.

Replenishing β Cells

As detailed in this disclosure, using lineage tracing, it has been demonstrated that long-term administration of low-dose GE is able to augment differentiation of pancreatic Sox9⁺ ductal cells into insulin-producing β cells in non-autoimmune diabetic mice with medium hyperglycemia. It has also been shown that although hyperglycemia is required for initiating the differentiation of Sox9⁺ ductal cells into insulin-producing β cells, medium hyperglycemia combined with long-term but not short-term administration of GE is required for an effective differentiation and reversal of diabetes.

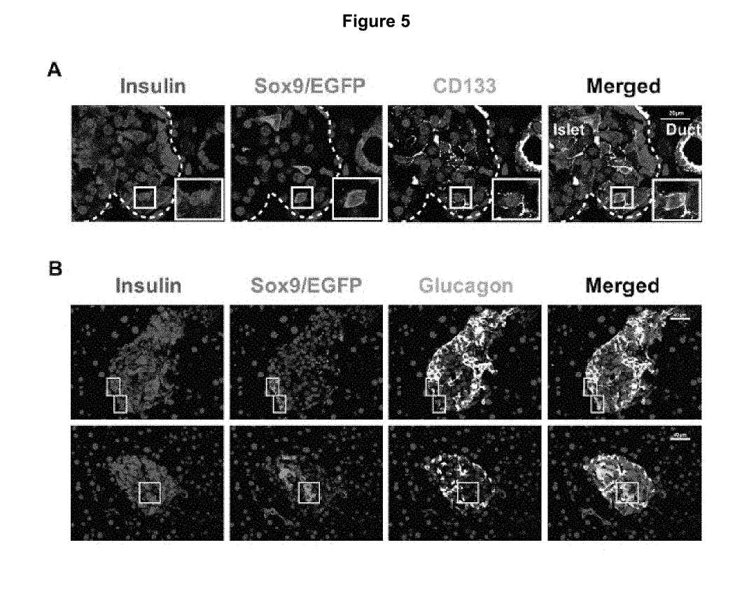

Previous reports showed that, during the embryonic development period, the pancreatic Sox9⁺ ductal cells differentiated into exocrine acinar cells and endocrine cells, including insulin-producing β cells (14-17); but whether or not this is the case in neonates or in adult mice after pancreas injury remains controversial (5, 14, 16, 17), and the cause of controversy remains unclear (29). It is demonstrated in this disclosure that long-term administration of GE under medium hyperglycemia condition is able to induce differentiation of Sox9⁺ ductal cells into insulin-producing β cells, as indicated by the co-existence of Sox9⁺ Ins⁺Glu⁺ and Sox9⁺ Ins⁺Glu⁻ cells in the islets (FIG. 5). This condition appears to be preferable, as long-term administration of GE under high hyperglycemia did not achieve the desired therapeutic effect. This may be due to the glucose toxicity to newly-generated 13 cells, as previously reported (19, 20). The combination of medium hyperglycemia and short-term administration of GE did not effectively augment Sox9⁺ ductal cell differentiation either, although the treatment effectively augmented the replication of pre-existing β cells. This may be due to the fact that the maturation of β cells derived from Sox9⁺ ductal cells is slower than replication of mature β cells, as indicated by previous publications (13). This observation may provide an explanation for previous publications showing that Sox9⁺ ductal cells and other pancreatic ductal cells did not differentiate into β cells in Alloxan-induced diabetic mice after one week treatment of GE (14); this may also provide an explanation for the lack of Sox9⁺ ductal-derived β cells in STZ-induced diabetic mice with high hyperglycemia (about 500 mg/dL) and in the absence of administration of growth factors (16, 18). Therefore, neogenesis from ducts is influenced by the type and extent of pancreatic injury as well as dependent on the affected cell types (30, 31).

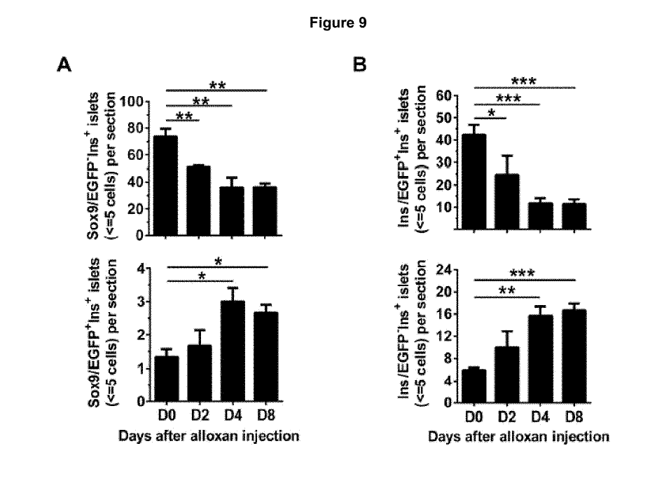

Hybrid Sox9⁺ periportal hepatocytes were recently reported to have high regenerative capacity (32). Sox9⁺ Ins⁺ pre-existing β cells may also be a type of hybrid cells that can have high regenerative capacity and contribute to β cell regeneration in the GE-treated diabetic mice. However, the contribution may be moderate, based on preliminary results shown in FIG. 9.

Hyperglycemia induces mature β cells to replicate (33). Chronic hyperglycemia is also toxic to β cells and causes their dysfunction and dedifferentiation (19, 20). But the impact of hyperglycemia on β cell neogenesis remains unclear. It is disclosed herein that hyperglycemia alone was able to increase the numbers of small islets from neogenesis, indicating that hyperglycemia may induce differentiation of Sox9⁺ ductal cells or expansion of Sox9⁺ Ins⁺ ductal epithelia cells. However, pancreatic epithelial cells do not usually express Glut2/Glucokinase machinery for glucose recognition (34). Long-term administration of GE under medium hyperglycemia but not under high hyperglycemia was able to effectively augment the differentiation of Sox9⁺ ductal epithelial cells into β cells.

It is also disclosed herein that there were hardly any Sox9⁺ Ins⁺ cells among ductal epithelial cells in normal control mice. Medium hyperglycemia induced the presence of Sox9/EGFP⁺Ins⁺ cells among ductal epithelial cells and GE treatment increased the frequencies of the Sox9/EGFP⁺Ins⁺ cells. Medium hyperglycemia condition is normally met in T1 D patients whose blood glucose level is controlled by administration of insulin. It is within the purview of one of ordinary skill in the art to adjust the blood glucose level of T1 D patients by adjusting the dose and/or dosage of insulin to optimize the therapeutic results of replacing/replenishing β cells. For example, the blood glucose level of a T1 D patient having high hyperglycemia is adjusted to 250-300 mg/dL by administration of more doses and/or a higher dosage of insulin to avoid the toxic condition for newly generated β cells.

In summary, the Examples described below demonstrate that pancreatic Sox9⁺ ductal cells in adult mice can differentiate into insulin-producing β cells and contribute to reversal of diabetes in non-autoimmune mice in the presence of medium hyperglycemia and long-term administration of GE, although Sox9⁺ ductal cells may only be one of the sources for β cell neogenesis in the diabetic mice. These studies also indicate that β cell neogenesis from Sox9⁺ ductal cells is a slow process and requires long-term growth factor therapy; additionally, adjustment of hyperglycemia to a medium level is also critical for the optimal therapeutic effect.

GE is administered to a subject for an extended period of time to allow differentiation of Sox9⁺ progenitors into β cells in vivo. It is within the purview of one of ordinary skill in the art to optimize the period of GE administration. In some embodiments, GE is administered to a subject for at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least 51 weeks, or at least 52 weeks. Depending on the therapeutic needs, it is also possible to extend the GE treatment to more than one year, such as 15 months, 18 months, 21 months or 24 months. In certain embodiments, the frequency of the GE treatment is a daily treatment, although one of skill in the art may adjust the frequency of the treatment as needed in accordance with monitoring of the subject recipient.

GE is administered to a subject at a low dose. For example, a low dose of gastrin for mice is about 1 μg/kg body weight, about 2 μg/kg body weight, about 3 μg/kg body weight, about 4 μg/kg body weight, or about 5 μg/kg body weight; a low dose of EGF is about 0.5 μg/kg body weight, about 1 μg/kg body weight, about 1.5 μg/kg body weight, or about 2 μg/kg body weight. In general, the dosage of GE in mice is lower than that in human. One of ordinary skill in the art can adjust the human dosage of GE based on the mouse dosage.

In some embodiments, gastrin and EGF are administered simultaneously to a subject. In other embodiments, gastrin and EGF are administered sequentially to a subject. When administered sequentially, one of ordinary skill in the art can adjust the interval between administrations to achieve a desired effect.

GE is administered shortly before, during, or shortly after administration of exogenous Sox9⁺ cells. Alternatively, GE is administered to a subject before administration of exogenous Sox9⁺ progenitors such that GE induces differentiation of endogenous Sox9⁺ cells into β cells before exogenous Sox9⁺ cells are supplemented to the subject.

The methods for replacement/replenishment of pancreatic β cells described herein may be performed on their own or in combination with a method of inducing mixed chimerism to reverse an autoimmune reaction.

For example, according to certian embodiments, exogenous Sox9⁺ cells are administered to a subject during induction of mixed chimerism of the subject. For example, Sox9⁺ cells from a donor may be administered to the subject along with donor hematopoietic stem cells (also referred to herein as bone marrow stem cells) during hematopoietic cell transplantation (HCT). In such embodiments, the exogenous Sox9⁺ cells are co-infused during administration of donor bone marrow stem cells, one or more population of conditioning donor cells, or both. In some embodiments, the exogenous Sox9⁺ cells are co-infused with one or more population of conditioning donor cells during HCT such that Sox9⁺ cells differentiate into β cells in vivo while the conditioning donor cells help induce mixed chimerism in the subject. Methods for inducing mixed chimerism that may be used in conjunction with or in combination with the methods for replenishing pancreatic β cells are described in detail below.

It is within the purview of one of ordinary skill in the art to select a suitable route of administration of GE and/or Sox9⁺ cells eripheral. For example, gastrin and EGF can be administered by oral administration including sublingual and buccal administration, and parenteral administration including intravenous administration, intramuscular administration, and subcutaneous administration. In a preferred embodiment, gastrin and EGF are administered intravenously. In some embodiments, gastrin and EGF are administered simultaneously. In certain embodiments, Sox9⁺ cells are administered by injection.

Inducing Mixed Chimerism

Also disclosed herein are various conditioning regimens for use in methods for establishing stable mixed chimerism in a subject without inducing GVHD. Induction of mixed chimerism in a subject was disclosed in U.S. Provisional Patent Application No. 62/253,657, entitled “Conditioning Regimens and Methods for Inducing Mixed Chimerism,” filed on Nov. 10, 2015, the content of which is incorporated herein by reference in its entirety and attached as Appendix A to this application.

According to the embodiments described herein, a conditioning regimen for use in the methods described herein includes one or more doses of cyclophosphamide (CY), pentostatin (PT), and anti-thymocyte globulin (ATG), administered individually or in combination to condition a recipient in preparation for and prior to transplantation of donor bone marrow cells.

The conditioning regimens described herein may further comprise administration of a population of donor conditioning cells that facilitate engraftment during HCT. The population of conditioning cells may include, but are not limited to, one or more of sorted donor CD8⁺ T cells, CD4⁺ T-depleted spleen cells and G-CSF-mobilized peripheral blood mononuclear cells. The population of donor conditioning cells may be administered on a day prior to an HCT procedure, or may be administered on the same day as the transplantation of donor bone marrow cells.

In some embodiments, the donor bone marrow cells may be a native population of bone marrow cells, while in other embodiments, the donor bone marrow cells may be a population of CD4+ T-depleted bone marrow cells. In embodiments where the donor bone marrow cells are a population of CD4+ T-depleted bone marrow cells, the conditioning regimen may optionally include administration of a population of conditioning cells, such as those described above.

In some embodiments, the donor conditioning cells, the donor bone marrow cells, or both may be HLA- or MHC-matched.

In other embodiments, the donor conditioning cells, the donor bone marrow cells, or both may be HLA- or MHC-mismatched. Recent studies indicate that induction of MHC-mismatched mixed chimerism may play an important role in the therapy of autoimmune diseases and conditions as well as in organ transplantation immune tolerance. Thus, according to certain embodiments described herein, an HLA- or MHC-mismatched or haploidentical donor may be desirable to avoid disease susceptible loci.

As detailed in this disclosure, EAE SJL/J mice conditioned with a combination of low-dose CY, PT, and ATG and then transplanted with CD4⁺ T-depleted spleen cells and bone marrow cells from MHC-mismatched C57BL/6 donors induced stable mixed chimerism. Induction of MHC-mismatched mixed chimerism is able to eliminate spinal cord tissue infiltration and augment regeneration of myelin sheath and cure acute phase EAE. Surprisingly, induction of MHC-mismatched mixed chimerism not only augments thymic deletion of host-type CD4⁺CD8⁺ thymocytes but also dramatically increases the percentage of Foxp3⁺ Treg cells among the host-type CD4⁺CD8⁺ thymocytes. Moreover, induction of MHC-mismatched mixed chimerism is not able to prevent EAE relapse in thymectomized recipients, even though there is expansion of host-type Treg cells in the periphery.

The term “low dose” as used herein refers to a dose of a particular agent, such as cyclophosphamide (CY), pentostatin (PT), or anti-thymocyte globulin (ATG), and is lower than a conventional dose of each agent used in a conditioning regimen, particularly in a myeloablative conditioning regimen. For example, the dose may be about 5%, about 10%, about 15%, about 20% or about 30% lower than the standard dose for conditioning. In certain embodiments, a low dose of CY may be from about 30 mg/kg to about 75 mg/kg; a low dose of PT is about 1 mg/kg; and a low dose of ATG may be from about 25 mg/kg to about 50 mg/kg. In general, different animals require different doses and human doses are much lower than mouse doses. For example, a low dose for BALB/c mice is about 30 mg/kg, for C57BL/6 mice is from about 50 mg/kg to about 75 mg/kg or from about 50 mg/kg to about 100 mg/kg, and for NOD mice is about 40 mg/kg.

In some embodiments, the human dose of CY used in the conditioning regimens and methods described herein may be from about 50 mg to about 1000 mg, from about 100 mg to about 800 mg, from about 150 mg to about 750 mg, from about 200 mg to about 500 mg, about 100 mg, about 200 mg, about 300 mg, about 400 mg, about 500 mg, about 600 mg, about 700 mg, or about 800 mg. In some embodiments, the human dose of ATG used in the conditioning regimens and methods described herein may be from about 0.5 mg/kg/day to about 10 mg/kg/day, from about 1.0 mg/kg/day to about 8.0 mg/kg/day, from about 1.5 mg/kg/day to about 7.5 mg/kg/day, from about 2.0 mg/kg/day to about 5.0 mg/kg/day, about 0.5 mg/kg/day, about 1.0 mg/kg/day, about 1.5 mg/kg/day, about 2.0 mg/kg/day, about 2.5 mg/kg/day, about 3.0 mg/kg/day, about 3.5 mg/kg/day, about 4.0 mg/kg/day, about 4.5 mg/kg/day, or about 5.0 mg/kg/day. In some embodiments, the human dose of PT used in the conditioning regimens and methods described herein may be from about 1 mg/m²/dose to about 10 mg/m²/dose, from about 2 mg/m²/dose to about 8 mg/m²/dose, from about 3 mg/m²/dose to about 5 mg/m²/dose, about 1 mg/m²/dose, about 2 mg/m²/dose, about 3 mg/m²/dose, about 4 mg/m²/dose, about 5 mg/m²/dose, about 6 mg/m²/dose, about 7 mg/m²/dose, about 8 mg/m²/dose, about 9 mg/m²/dose, or about 10 mg/m²/dose.

In another aspect, the conditioning regimens and methods described herein include administering the CY, PT, and/or ATG on a daily, weekly, or other regular schedule. For example, administration of CY may be daily; administration of PT may be weekly or at an interval greater than every day (e.g., every two or three days); and administration of ATG may be daily, weekly, or at an interval greater than every day (e.g., every two or three days).

In certain embodiments, a dose of CY may be administered to the recipient on a daily basis for up to about 28 days, up to about 21 days, up to about 14 days, up to about 12 days or up to about 7 days prior to transplantation. In certain embodiments, a dose of CY may be administered to the recipient every other day for up to about 28 days, up to about 21 days, up to about 14 days, or up to about 7 days prior to transplantation. In one example, a dose of CY may be administered to the recipient on a daily basis for about 21 days prior to transplantation.

In certain embodiments, a dose of PT may be administered to the recipient every day, every other day, every third day, every fourth day, every fifth day, every sixth day, or every week for up to about 28 days, up to about 21 days, up to about 14 days, up to about 12 days or up to about 7 days prior to transplantation. In one example, a dose of PT may be administered to the recipient every week for about 21 days prior to transplantation.

In certain embodiments, a dose of ATG may be administered to the recipient every other day, every third day, every fourth day or every fifth day for up to about 28 days, up to about 21 days, up to about 14 days, up to about 12 days or up to about 7 days prior to transplantation. For example, a dose of ATG may be administered to the recipient every third day for about 21 days prior to transplantation. In certain embodiments, a dose of ATG may be administered for two, three, or four days in a row about one 7 days, 8 days, 9 days, 10 days, 11 days, 12 days, 13 days, or 14 days prior to transplantation.

In one embodiment, the conditioning regimen includes (i) three doses of PT at a dose of about 4 mg/m²/dose may be administered to a human patient about 3 weeks, about 2 weeks and about 1 week before transplantation; (ii) three doses of ATG at a dose of about 1.5 mg/kg/day may be administered to a human patient about 12 days, about 11 days, and about 10 days before transplantation; and (iii) CY at a dose of about 200 mg orally may be administered to a human patient on a daily basis about 3 weeks before transplantation.

It is within the purview of one of ordinary skill in the art to select a suitable route of administration of CY, PT and ATG. For example, these agents can be administered by oral administration including sublingual and buccal administration, and parenteral administration including intravenous administration, intramuscular administration, and subcutaneous administration. In a preferred embodiment, one or more of CY, PT and ATG are administered intravenously. In some embodiments, CY is administered orally and ATG and PT are administered intravenously.

The depletion of CD4⁺ cells and/or the combination of CY, PT and ATG allows lowering the dose of each of CY, PT and ATG, thereby to reduce the toxic side effects while achieving mixed chimerism. It is within the purview of one of ordinary skill in the art to adjust the dose of each of CY, PT and ATG to achieve the desired effect.

Mixed chimerism may be induced by conditioning with the combination of CY, PT and ATG and supplying to the recipient donor bone marrow cells and donor CD8⁺ T cells that facilitate engraftment. In some embodiments, the methods disclosed herein may include transplantation of CD4⁺ T-depleted bone marrow cells following administration of CY, PT and ATG in accordance with the conditioning regimens described above. In some embodiments, the methods disclosed herein may include administering donor bone marrow cells, and one or more types of cells selected from CD4⁺ T-depleted spleen cells, donor CD8⁺ T cells, and donor G-CSF-mobilized peripheral blood mononuclear cells following administration of the CY, PT and ATG.

In another aspect, the disclosure provided herein relates to a method of inducing stable mixed chimerism in a recipient by administration of radiation-free, low doses of CY, PT and ATG, followed by transplantation of CD4⁺ T-depleted bone marrow cells. In certain embodiments, mixed chimerism in a recipient is induced by administration of radiation-free, low doses of CY, PT and ATG, a therapeutically effective amount of donor bone marrow cells, and a therapeutically effective amount of one or more types of cells selected from donor CD4⁺ T-depleted spleen cells, donor CD8⁺ T cells, and donor G-CSF-mobilized peripheral blood mononuclear cells. CY, PT and ATG are administered to the recipient before transplantation, in accordance with the conditioning regimen described above. In some embodiments, the donor cells are MHC- or HLA-matched. In preferred embodiments, the donor cells are MHC- or HLA-mismatched. In certain embodiments, the mixed chimerism is HLA- or MHC-mismatched mixed chimerism.

Pharmaceutical Compositions and Conditioning Regimens

According to some embodiments, the agents and/or cells administered to a subject may be part of a pharmaceutical composition and/or a conditioning regimen comprising one or more pharmaceutical compositions that are administered in combination or in conjunction with each other, and can be administered simultaneously or at different times in accordance with the embodiments described herein. Each pharmaceutical composition used in the methods described herein may include one or more of CY, PT, ATG, gastrin and EGF and a pharmaceutically acceptable carrier; or one or more populations of donor cells and a pharmaceutically acceptable carrier. Based on suitable administration schedule and/or administration route, it is within the purview of one of ordinary skill in the art to combine one or more agents in a composition. For example, a pharmaceutical composition may include gastrin and EGF, along with one or more pharmaceutically acceptable carriers. In another example, a pharmaceutical composition may further include one or more of CY, PT, and ATG.

The pharmaceutical compositions described herein may include compositions including a single agent or a single type of donor cell (e.g., donor Sox9⁺ cells, donor bone marrow cells, donor CD4⁺ T-depleted spleen cells, donor CD8⁺ T cells, or donor G-CSF-mobilized peripheral blood mononuclear cells) in each composition, or alternatively, may include a combination of agents, populations of cells, or both.

One or more populations of cells may be combined in one pharmaceutical composition. In some embodiments, the pharmaceutical composition may include Sox9⁺ cells and bone marrow stem cells from a donor. In some embodiments, the pharmaceutical composition may further include Sox9⁺ cells and one or more types of cells selected from CD4⁺ T-depleted spleen cells, donor CD8⁺ T cells, and donor G-CSF-mobilized peripheral blood mononuclear cells.

The pharmaceutical compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

In one embodiment, the pharmaceutically acceptable carrier is an aqueous carrier, e.g. buffered saline and the like. In certain embodiments, the pharmaceutically acceptable carrier is a polar solvent, e.g. acetone and alcohol.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### EXAMPLES

Materials and Methods

Mice. Wild type C57BL/6, breeders of Ins1^{CreERT} mice were purchased from The Jackson Laboratory. Breeders of Sox9^{CreERT2} mice were previously described (17, 18) and provided by M. Sander's lab at UCSD. For the following examples, a founder that induces recombination in cells with high SOX9 expression was used, and higher recombination efficiency than the founder was described previously (17, 18). ROSA26^{mT/mG} breeders are provided by C. Chen's lab at City of Hope (COH). All mice were maintained in a pathogen-free room in City of Hope Animal Research Center. The experimental procedures were approved by COH Institutional Animal Care and Use Committee (IACUC).

Other Materials.

Tamoxifen, human recombinant EGF and human [Leu¹⁵]-Gastrin I were purchased from Sigma. Histology and immunofluorescence staining, morphometric analysis and cell counting, Intraperitoneal Glucose Tolerance Test (IPGTT), pancreas isolation, intracellular staining and flow cytometry, and statistical analysis are described below.

Induction of diabetes by Alloxan and in vivo activation of reporter genes by Tamoxifen.

8-week-old female mice were I.V. injected with β cell toxin Alloxan (Sigma) at 70 mg/kg body weight. Tail vein blood glucose was measured daily or twice a week with Precision Xtra Glucose Meter (Abbott Diabetes Care Inc.) with a maximum reading of 500 mg/dL Tamoxifen (TM, Sigma) was prepared at 20 mg/mL in corn oil (Sigma). For tracing studies in adult mice, a total of 20 mg of TM was given intravenously in five doses (each 4 mg) over a 2-week period. Mice rendered diabetic (>300 mg/dL) over a period of 28 days were used in subsequent experiments.

Preparation and Injection of Growth Factors.

For low-dose growth factor injection, human recombinant EGF (Sigma) was dissolved in sterile 10 mmol/L acetic acid solution at a stock concentration of 3 μg/mL. Human [Leu¹⁵]-Gastrin I (Sigma) was dissolved in phosphate-buffered saline (PBS) to a stock concentration of 3 μg/mL. The stocks were stored in aliquots at −80° C. The stocks were diluted in sterile PBS (pH7.4) to a working concentration of EGF (1 μg/kg body weight) and Gastrin (3 μg/kg body weight). PBS vehicle (control) or Gastrin plus EGF (GE) were intraperitoneally administered to the mice daily for 56 days.

For high-dose growth factors treatment, human recombinant EGF was dissolved in sterile 10 mmol/L acetic acid solution at a stock concentration of 1 mg/mL. Human [Leu¹⁵]-Gastrin I was dissolved in phosphate-buffered saline (PBS) to a stock concentration of 0.5 mg/mL. The mixture was injected into mini-osmotic pumps (Alzet 1007D) to obtain a flux rate of 3 μg/kg body weight per hour for Gastrin and 10 μg/kg body weight per hour for EGF. Pumps with growth factors or vehicle composed of 5 mmol/L acetic acid solution were implanted intraperitoneally at day 28 post Alloxan injection. These pumps release their content for approximately 7 days.

Histology and Immunofluorescence Staining.

Samples for immunofluorescence analysis were fixed overnight in 10% formalin or 4% paraformaldehyde (PFA), embedded in paraffin and cut to 4-5 μm tissue sections. Antigen retrieval in paraffin sections was heat-mediated with Antigen Unmasking Solution, Citric Acid Based (Vector Labs). Primary antibodies include guinea pig anti-insulin (1:4000; DAKO), mouse anti-glucagon (1:1000; Sigma), goat anti-GFP conjugated FITC (1:100; Abcam), rabbit anti-Sox9 (1:1000; Millipore), rabbit anti-Pdx1 (1:1000; Abcam), rabbit anti-Nkx6.1 (1:100; Abcam) and rat anti-CD133 (1:100; eBioscience). Secondary antibodies for indirect fluorescent staining conjugated to AMCA, Cy3 or AlexaFluor 647 were obtained from Jackson ImmunoResearch Labs. The antibodies conjugated to AMCA were used at 1:100, and the antibodies conjugated to Cy3 or AlexaFluor 647 were used at 1:1000. Nuclei were labeled by DAPI (1 μg/mL; Sigma). Tissue sections were viewed on an Olympus IX81 Automated Inverted Microscope. Images were captured and processed using Image-Pro Premier.

Morphometric Analysis and Cell Counting.

For morphometric analyses, the entire adult pancreas was kept flat and totally sectioned horizontally. Thinner sections were taken at 20 μm intervals throughout the organ. Four sections from different levels (˜2% of the pancreas) in one adult mouse were analyzed.

Percentage of EGFP⁺Sox9⁺ cells was determined by the number of EGFP⁺Sox9⁺ cells divided by the total number of Sox9⁺ cells. Cells were counted in ten random fields of view of a tissue section. To quantify the number of lineage-labeled Insulin⁺ cells, all Insulin⁺ cells and Ins/EGFP⁺Ins⁺ cells on a section were counted, and then the percentage of Ins/EGFP⁺Ins⁺ cells amongst total Insulin⁺ cells was determined. To quantify the number of lineage-labeled Insulin⁺ cells arising from Sox9⁺ cells, all Insulin⁺ cells and Sox9/EGFP⁺Ins⁺ cells on a section were counted, and then the percentage of Sox9/EGFP⁺Ins⁺ cells among total Insulin⁺ cells was determined. To quantify Sox9/EGFP⁺Ins⁺ cells among ductal tubes, all Sox9/EGFP⁺ ductal cells co-expressing insulin in ducts were counted on a section. For β cell surface measurements, four sections per mouse were selected for histochemical staining of insulin. The Insulin⁺ area of the entire section was captured using Hamamatsu Nanozoomer 2.0 HT. The percentage of Insulin⁺ area was determined by Insulin⁺ area divided by total pancreatic tissue area in a slide, using Image-Pro Premier software. In all of the morphometric analysis and cell counting, four sections from different levels with 20 μm intervals in each mouse were counted. Average count of each mouse was calculated. Mean±SEM from at least 4 mice in each group is presented.

Intraperitoneal Glucose Tolerance Test (IPGTT).

Mice were fasted for 16 hours and injected intraperitoneally (i.p.) with glucose (2 g/kg body weight). Blood glucose concentration was measured from tail vein blood with Glucose Meter. Insulin concentration in plasma from time points at 0 and 10 min after glucose injection was determined with the Mouse Insulin ELISA kit (Mercodia).

Pancreas Isolation, Intracellular Staining and Flow Cytometry.

The dissected pancreas was digested and dissociated to single cells with collagenase B (2-4 mg/mL, Roche) and DNase I (2,000 U/mL per pancreas) (Roche), refer to Jin et al (29). For flow cytometry analysis, the cell suspension was first incubated with anti-mouse CD16/32 (1:25; eBioscience) and Live/Dead (1:1000; Life Technology) for 5 minutes on ice to diminish nonspecific binding. APC-conjugated anti-mouse CD133 (1:100; eBioscience) was added, and the cells were incubated on ice for 20 minutes. After washing twice, cells were immediately fixed and permeabilized (4% PFA, 0.1% saponin/PBS, 30 min) on ice. After fixation, intracytoplasmic staining was performed with rabbit anti-insulin conjugated AlexaFluor647 (1:50; Bioss), goat anti-GFP conjugated FITC (1:100; Abcam) and mouse monoclonal IgG1 anti-glucagon (Sigma-Aldrich). Simultaneous staining using mouse IgG1 antibodies required Invitrogen's Zenon pre-labeling technology (Pacific Blue). After the final wash in 0.1% saponin/PBS, cells were post-fixed in 2% PFA and immediately analyzed with flow cytometry, a CyAn immunocytometry system (Dako Cytomation, Fort Collins, Colo.), and the data was analyzed with FlowJo software (TreeStar, San Carlos, Calif.) as described in previous publications (4).

Statistical Analysis.

All quantitative data are shown as mean±SEM. All data were analyzed by using GraphPad software (Prism Version 6.0, GraphPad Software). Comparison of kinetic blood glucose change was evaluated with two-way ANOVA test. Comparison of means among multiple groups was evaluated with one-way ANOVA test. Comparison of two means was performed with an unpaired two-tailed Student t test.

### Example 1

This example demonstrates that long-term administration of low-dose GE augmented differentiation of pancreatic Sox9+ ductal cells into β cells with reversal of hyperglycemia.

This example investigates whether Sox9⁺ pancreatic ductal cells could give rise to insulin-producing β cells in adult diabetic mice after GE treatment, using non-autoimmune Sox9^{CreERT2}R26^{mT/mG} C57BL/6 mice. First, a β cell neogenesis mouse model in non-autoimmune diabetic C57BL/6 mice was established. 8 week-old adult female C57BL/6 mice were induced to develop diabetes by one intravenous injection of Alloxan (70 mg/kg body weight) as previously described (14, 27). Mice developed variable levels of hyperglycemia, including mild (<300 mg/dL), medium (300-450 mg/dL) and high hyperglycemia (>450 mg/dL). C57BL/6 mice (n=222) were induced to render diabetic by intravenously injecting one dose of Alloxan (70 mg/kg body weight) at day 0. Blood glucose (BG) concentrations in mice were measured twice a week for 4 weeks. As shown in Table 1, by day 28, 17% of the mice had mild hyperglycemia (<300 mg/dL), 45% had medium hyperglycemia (300-450 mg/dL) and 38% had high hyperglycemia (>450 mg/dL).

TABLE 1
Alloxan treatment induces diabetes with different
levels of blood glucose in C57BL/6 mice

| | Blood Glucose | | |
|---|---|---|---|
| | BG < 300 | 300 ≤ BG < 450 | BG ≥ 450 |
| | (mg/dL) | (mg/dL) | (mg/dL) |
| Glucose level | Mild | Medium | High |
| Mouse No. | 38 | 101 | 83 |
| Percentage | 17% | 45% | 38% |

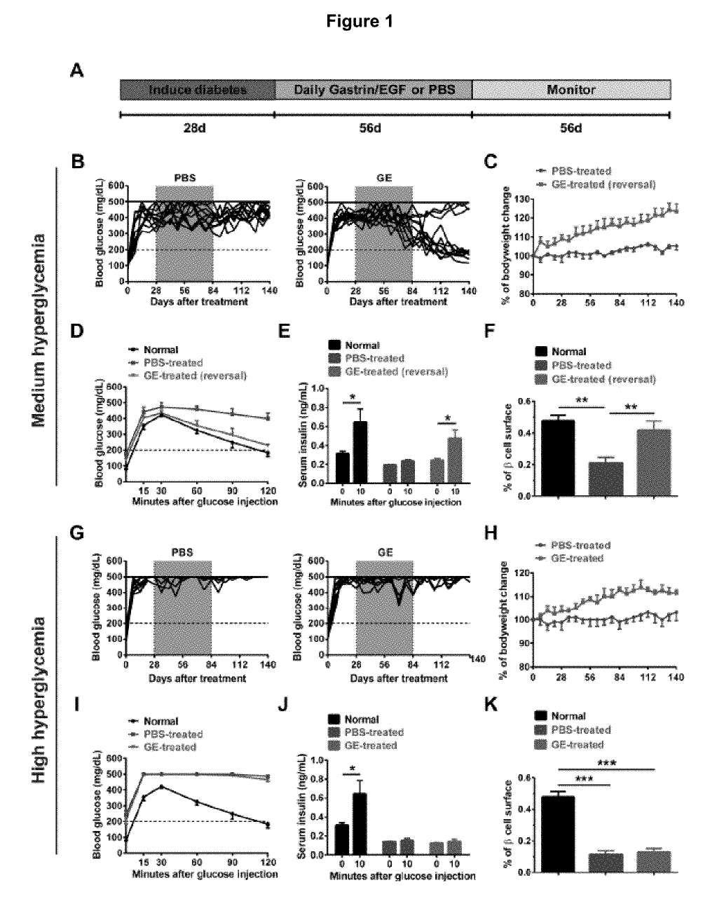

Since some diabetic mice with mild hyperglycemia recovered spontaneously, only diabetic mice with medium or high hyperglycemia were used in this study. As illustrated in FIG. 1A, diabetic mice with medium and high hyperglycemia were given a daily injection of low-dose GE (gastrin 3 μg/kg body weight, EGF 1 μg/kg body weight), as previously described (4, 26) or control PBS for 8 weeks, starting at 4 weeks after induction of diabetes. The treated mice were monitored for blood glucose for another 8 weeks before ending the experiments.

GE treatment gradually led to reversal of hyperglycemia in 75% (9/12) of diabetic mice with medium hyperglycemia, whereas no reversion was seen (12/12) in PBS-treated mice (P<0.01, FIG. 1B). As compared to PBS-treated diabetic mice, GE-treated mice with reversal of diabetes showed marked improvement in body weight growth (P<0.01, FIG. 1C), rapid blood glucose recovery during intraperitoneal glucose tolerance tests (IPGTT) (P<0.01, FIG. 1D), marked increase of serum insulin secretion during IPGTT (P<0.05, FIG. 1E), and increase of β cell surface, which reached levels similar to normal control mice (P<0.01, FIG. 1F). In contrast, GE treatment was not able to reverse diabetes in mice with high hyperglycemia (FIG. 1G). Although GE treatment was able to improve body weight growth (P<0.05, FIG. 1H), it was not able to augment blood glucose recovery or increase insulin production during fasting IPGTT, or increase β cell surface (FIGS. 1I-1K). These results indicate that GE treatment can augment 13 cell regeneration only in mice with medium hyperglycemia.

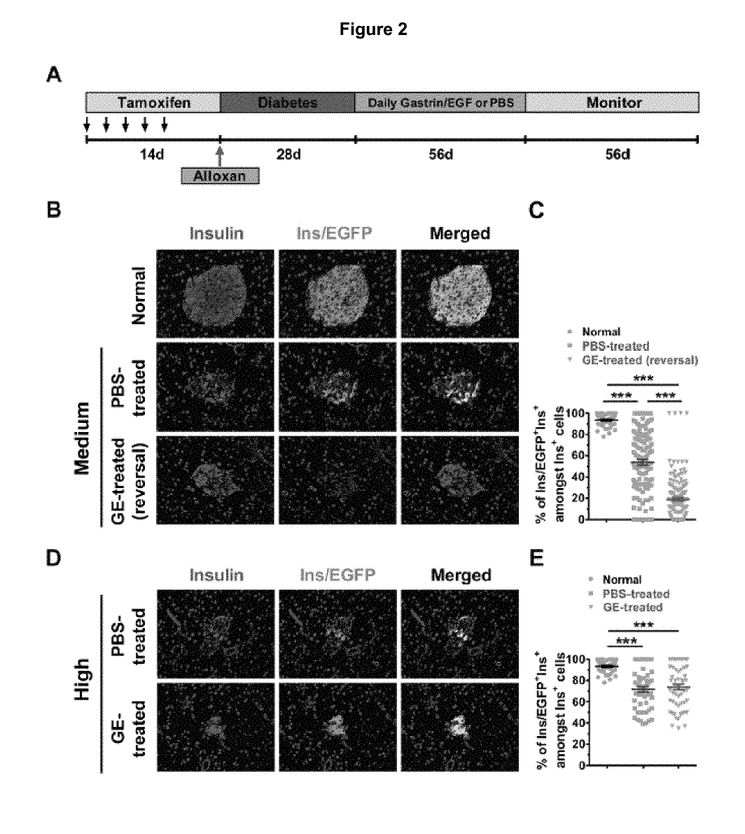

Next, lineage tracing was used to find out whether there is neogenesis of 13 cells in the GE-treated mice with reversal of diabetes. Accordingly, C57BL/6 mice with Ins1^{CreERT} R26^{mT/mG} transgene were treated with Tamoxifen™ to label the pre-existing insulin-producing β cells. Thereafter, the mice were induced to develop diabetes with Alloxan and then treated with GE and monitored for blood glucose as described in FIG. 2A. In non-diabetic mice, TM treatment labeled more than 90% of the pre-existing β cells in the islets with EGFP (FIG. 2B upper row and FIG. 2C), but only approximately 50% of the Insulin⁺ islet remnants in the pancreas of PBS-treated diabetic mice with medium hyperglycemia were labeled with EGFP, that is, EGFP⁺Insulin⁺ (P<0.01, FIG. 2B second row and FIG. 2C). The EGFP⁺Insulin⁺ islets and clusters in the GE-treated mice with reversal of diabetes were further reduced to ˜20% (FIG. 2B bottom row and FIG. 2C), which is markedly different from PBS-treated mice (P<0.001, FIGS. 2B and 2C). In addition, the Insulin⁺ clusters in the diabetic mice with high hyperglycemia after GE or PBS treatment were both ˜70% EGFP⁺, which was still significantly lower than that of control mice with normal glycemia (P<0.01, FIGS. 2D and 2E), even GE treatment did not change the percentage as compared to PBS treatment (FIG. 2E). These results indicate that hyperglycemia can induce β cell neogenesis and medium hyperglycemia combined with administration of low-dose GE can markedly augment the process.

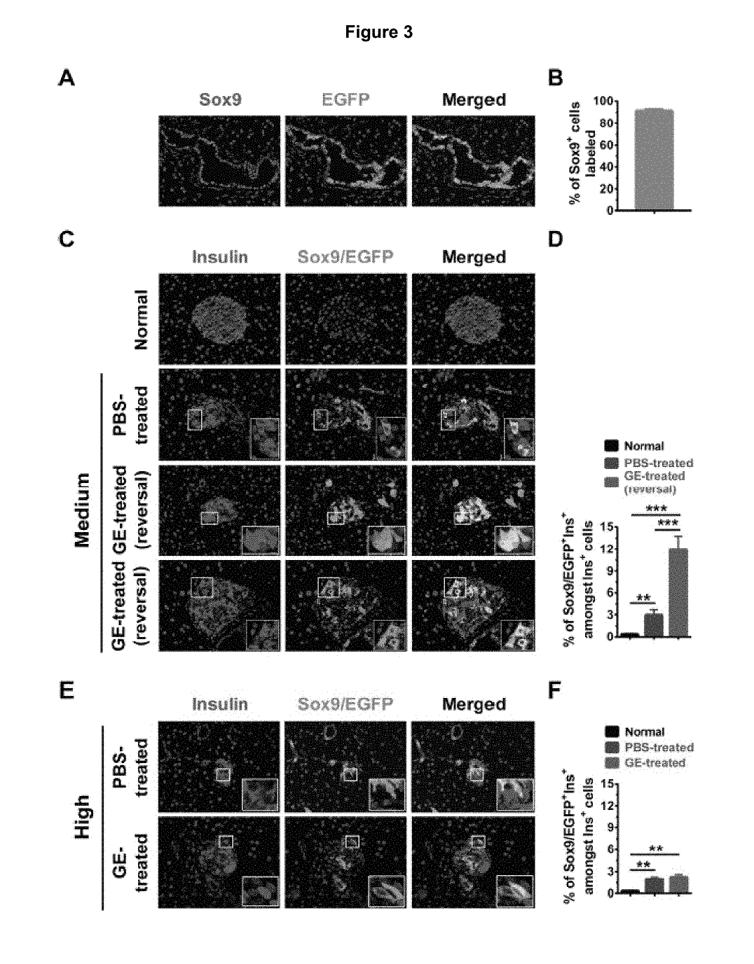

Next, Sox9^{CreERT2}R26^{mT/mG} mice were used to determine whether newly generated β cells in the GE-treated diabetic mice originate from Sox9⁺ cells in the pancreatic ducts. The mice were induced to develop diabetes and then treated with GE and monitored for blood glucose as described above (FIG. 2A). TM treatment was able to label over 90% of pancreatic Sox9-expression ductal epithelial cells with EGFP in Sox9^{CreERT2}R26^{mT/mG} C57BL/6 mice (FIGS. 3A and 3B). However, most of islets in the normal control did not have any Sox9/EGFP⁺Insulin⁺ cells (FIG. 3C, first row). The percentage of Sox9/EGFP⁺Insulin⁺ cells among total Insulin⁺β cells in normal control mice was less than 0.4% (FIG. 3D). There were scattered Sox9/EGFP⁺ cells among Insulin⁺ islet remnants or Insulin⁺ cell clusters in medium diabetic mice treated with PBS (FIG. 3C, second row). The percentage of Sox9/EGFP⁺Insulin⁺ cells among total Insulin⁺ cells was ˜3%, and it was a significant increase as compared to normal glycemia control mice (P<0.01, FIG. 3D).

In contrast, there were two types of islets or clusters in the GE-treated mice with reversal of diabetes (FIG. 3C, bottom two rows): one type is all Sox9/EGFP⁺Insulin⁺ cells; the other is a mixture of Sox9/EGFP⁺Insulin⁺ and Sox9/EGFP⁻Insulin⁺ cells. The percentage of Sox9/EGFP⁺ cells among total Insulin⁺ cells increased by more than 4-fold in the GE-treated mice as compared to PBS-treated control mice (P<0.01, FIGS. 3C and 3D). In addition, GE-treatment did not significantly increase the percentage of Sox9/EGFP⁺ cells among total Insulin⁺ cells in diabetic mice with high hyperglycemiawhen compared with PBS-treated control mice (FIGS. 3E and 3F). Taken collectively, these results indicate that hyperglycemia can induce differentiation of Sox9/EGFP⁺ pancreatic ductal cells into insulin-producing cells; GE treatment can augment this differentiation in diabetic mice with medium hyperglycemia but not in the mice with high hyperglycemia.

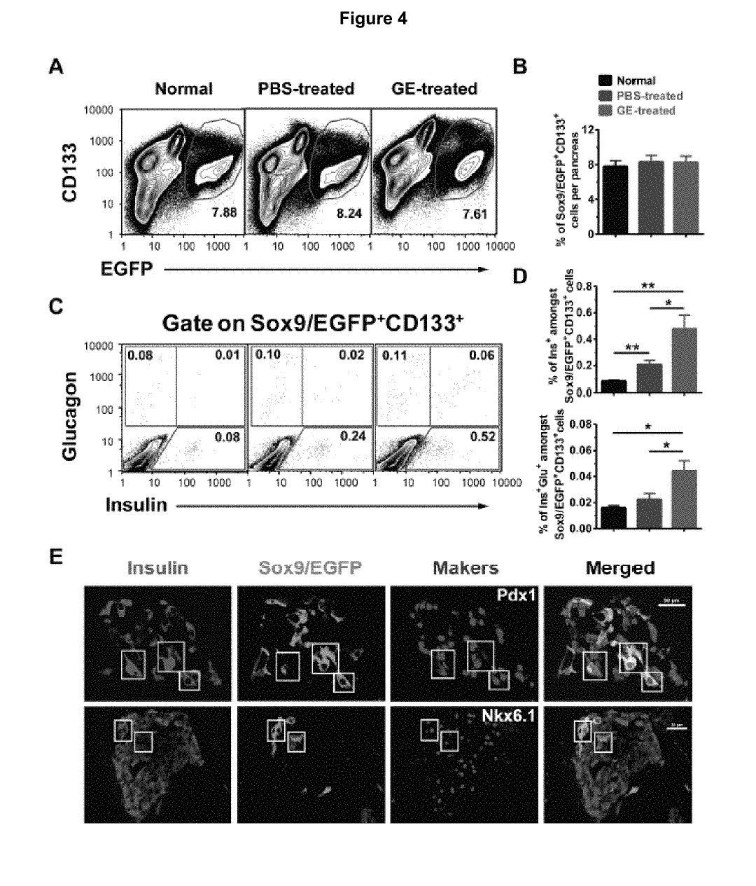

A previous report showed that Sox9-GFP⁺CD133⁺ but not Sox9-GFP⁺CD133⁻ cells contain cells that gave rise to insulin-producing colonies in an in vitro culture (23); therefore whether GE treatment increased the percentage of Sox9/EGFP⁺CD133⁺ ductal cells was tested using flow cytometry analysis. It was found that GE treatment did not expand the Sox9/EGFP⁺CD133⁺ cell population, since the percentage of Sox9/EGFP⁺CD133⁺ cells among total pancreatic cells was similar to control mice (FIGS. 4A and 4B). However, GE treatment significantly increased the percentage of Insulin⁺ cells among Sox9/EGFP⁺CD133⁺ cells, which appears to have lower levels of insulin expression)(insulin^{lo} when compared with PBS-treated or normal controls (FIGS. 4C and 4D). The Sox9/EGFP⁺CD133⁺Ins⁺ cells were also identified by histoimmunofluoresent staining (FIG. 5A). These results suggest that Sox9/EGFP⁺CD133⁺Ins⁺ cells may be newly generated β cells or Insulin⁺ progenitors.

β Cells Expressing Pdx1 and Nkx6.1 (12).

Consistently, the Sox9/EGFP⁺Ins⁺ cells in the GE-treated recipients also expressed Pdx1 and Nkx6.1 (FIG. 4E). In addition, although the percentage of Insulin⁺Glucagon⁺ cells among Sox9/EGFP⁺CD133⁺ cells was very low, there was a significant difference between GE-treated and PBS-treated recipients (P<0.05, FIGS. 4C and 4D). Sox9/EGFP⁺Ins⁺Glu⁺ cells were also clearly observed in the islets of GE-treated mice as judged with histoimmunofluorescent staining (FIG. 5B). Taken collectively, these results suggest that GE-treatment can augment differentiation of Sox9⁺ CD133⁺ cells into insulin-producing β cells.

### Example 2

This example demonstrates that medium hyperglycemia was required for effective augmentation of Sox9⁺ ductal cells differentiation into insulin-producing 13 cells during GE treatment.

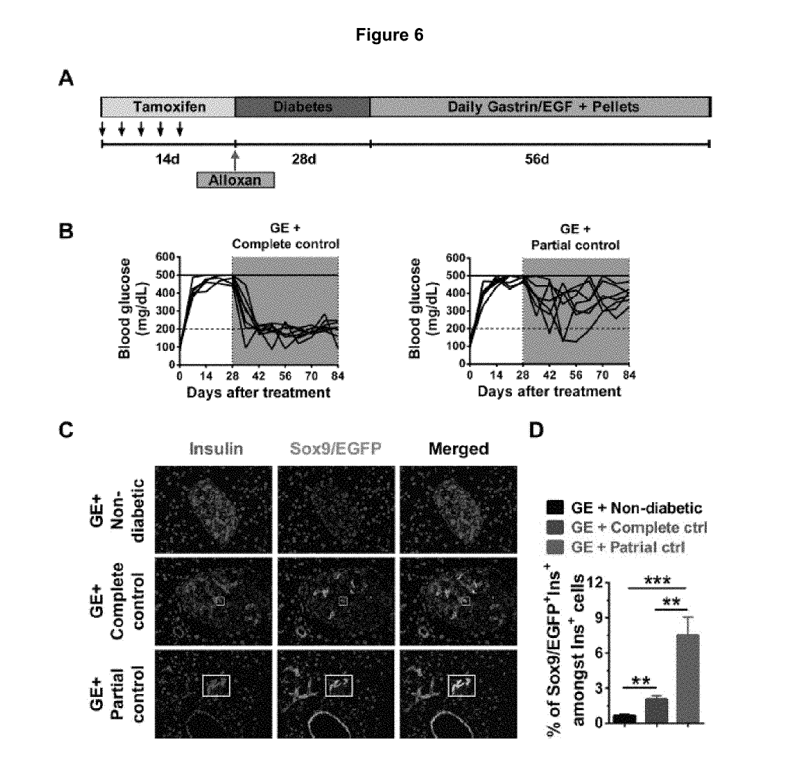

Since significant β cell neogenesis from Sox9⁺ ductal cells was observed in GE-treated diabetic mice with medium but not high hyperglycemia (FIG. 3), this example investigated whether medium hyperglycemia is required for augmenting 13 cell neogenesis from Sox9⁺ cells during GE treatment. Accordingly, the effect of GE treatment was compared in non-diabetic normal control mice and diabetic mice with high hyperglycemia under partial or complete normalization of hyperglycemia by implanting insulin pellets (FIG. 6A). GE-treatment hardly induced Sox9/EGFP⁺Ins⁺ cells in the islets of non-diabetic normal control mice (FIGS. 6C and 6D). As compared to complete control of blood glucose, partial normalization of blood glucose markedly increased β cells differentiated from Sox9/EGFP⁺ cells in diabetic recipients after GE treatment (P<0.01, FIGS. 6B-6D). These results indicate that medium hyperglycemia is required for effective augmentation of Sox9⁺ ductal cells differentiation into insulin-producing β cells during GE treatment.

### Example 3

This example demonstrates that short-term administration of high-dose GE did not augment β cell neogenesis from Sox9⁺ ductal cells in mice with medium hyperglycemia.

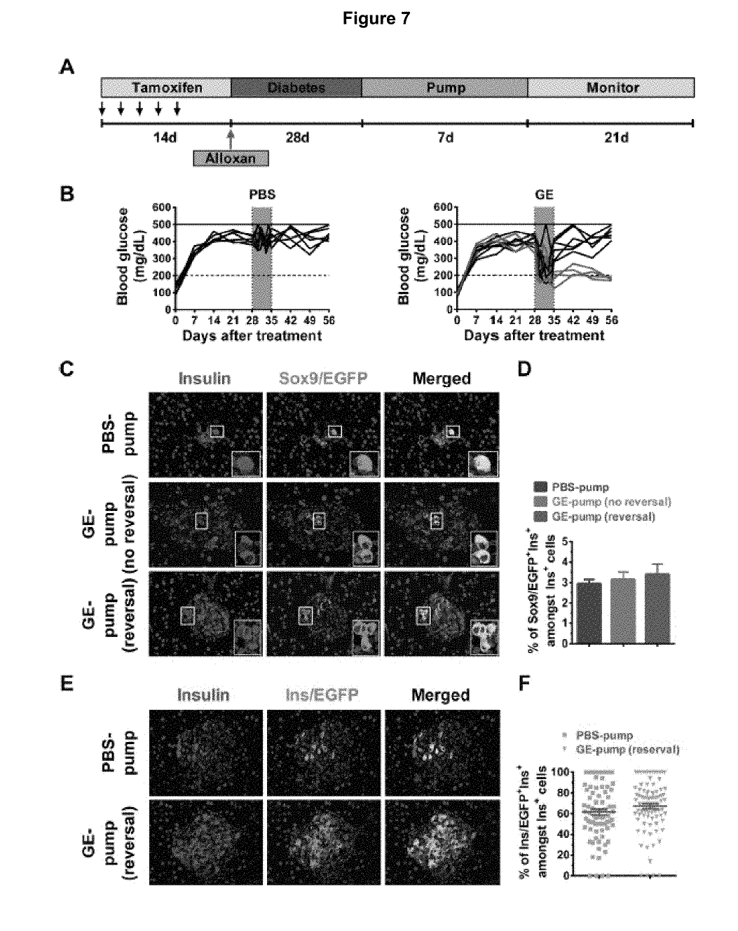

Short-term (one week) administration of high-dose GE has been reported to reverse Alloxan-induced diabetes in adult mice, although it was not able to induce Hnf1β⁺ ductal cell differentiation into β cells (14). Thus, this example investigated this issue in diabetic Sox9^{CreERT2}R26^{mT/mG} mice. Similar to the previous reports (14, 27) and in the diagram (FIG. 7A), GE was administered by implanting a pump containing Gastrin (release rate: 3 μg/kg body weight per hour) and EGF (release rate: 10 μg/kg body weight per hour) for release over 7 days, and then the blood glucose was monitored for 3 weeks. It was found that 40% (4/10) of GE-treated mice with medium hyperglycemia showed reversal of hyperglycemia, but none in PBS-treated control (P<0.01, FIG. 7B). Although there was a small percentage (˜3%) of Sox9/EGFP⁺Ins⁺ cells, GE treatment did not significantly increase the percentage between GE-treated mice with or without reversal (FIGS. 7C-7D).

Furthermore, it was found that, using Ins1^{CreERT}R26^{mT/mG} mice, the reversal of diabetes after short-term administration of high-dose GE was associated with replication of pre-existing β cells, as the percentage of Ins/EGFP⁺ pre-existing β cells among Insulin⁺ cells of islets in the short-term GE-treated mice was similar to that in the PBS-treated mice (FIGS. 7E and 7F). Taken collectively, although short-term administration of GE can augment pre-existing β cell replication, it cannot augment Sox9⁺ ductal cell differentiation into insulin-producing β cells, and long-term administration of GE is required for augmenting the Sox9⁺ ductal cell differentiation.

### Example 4

This example demonstrates that long-term administration of low-dose GE augmented differentiation of Sox9+ cells in the pancreatic ducts into insulin-producing β cells.

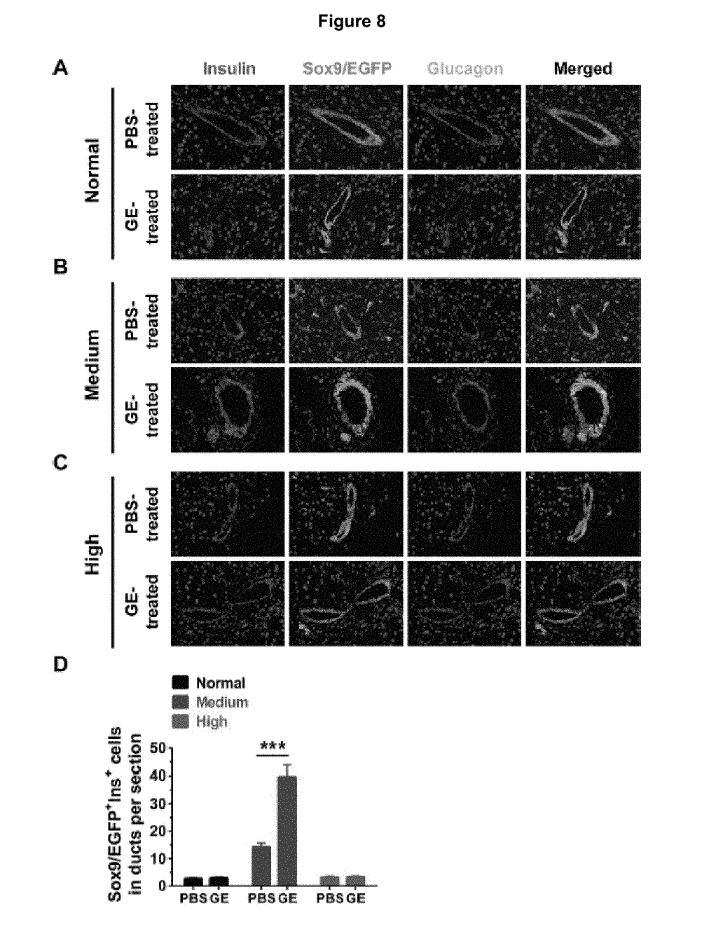

It was observed that there were few Sox9/EGFP⁺Ins⁺ cells among pancreatic ductal epithelial cells in the normal non-diabetic adult mice, as judged by anti-insulin immunofluorescent staining; there were less than 3 cells per section, and GE treatment did not show any significant increase (FIGS. 8A and 8D). However, as compared to normal non-diabetic adult mice, medium hyperglycemia alone significantly increased the Sox9/EGFP⁺Ins⁺ cells among ductal epithelial cells, which reached about 15 cells per section (P<0.01), although high hyperglycemia alone did not increase the number, as compared to non-diabetic normal control (FIGS. 8B-8D). GE-treatment significantly increased Sox9/EGFP⁺Ins⁺ cells in medium hyperglycemic mice, which reached about 40 cells/section (P<0.001), but GE-treatment did not increase the number at all in high hyperglycemic mice (FIGS. 8B-8D). Taken together, some Sox9⁺ cells in the pancreatic ducts could become Insulin⁺ cells before budding off to form islets, and medium hyperglycemia and GE-treatment increases the frequencies of Sox9⁺ Ins⁺ cells among ductal epithelial cells and augments their differentiation into insulin-producing islet β cells.

### Example 5

This example demonstrates induction of stable mixed chimerism and differentiation of Sox9⁺ cells into insulin-producing β cells can be achieved.

As described in Appendix A, a radiation-free conditioning regimen with low doses of cyclophosphamide (CY), pentostatin (PT), and anti-thymocyte globulin (ATG), can induce induced mixed chimerism without causing GVHD in EAE mice. Such an induction of mixed chimerism can be used to eliminate an autoimmune response in a subject suffering from a condition resulting from that autoimmune response. For example, a subject suffering from Type I diabetes lacks pancreatic 13 cells as a result of an autoimmune response. Accordingly, the embodiments described herein may be used in combination with the induction of mixed chimerism to eliminate the autoimmune response that is responsible for Type I diabetes and to replace and/or replenish insulin-producing β cells, thereby treating and/or reversing Type I diabetes. Data from a mouse study may be used to extrapolate such a combination treatment to human subjects.

Briefly, mouse studies may be performed as follows. Autoimmune type 1 diabetic NOD mice will be conditioned and induced for mixed chimerism with a regimen consisting of clinically available reagents including CY, PT and ATG. In brief, the mice may be conditioned with i.p. injection of CY (75 mg/Kg) daily for 12 days, PT (1 mg/Kg) every 4 days for a total 4 injections, and ATG (25 mg/Kg) every 4 days for 3 injections. The recipients conditioned with CY+PT or CY+ATG may be used as controls. On day 0, recipients undergo hematopoietic cell transplantation (HCT), wherein the recipients are transplanted with bone marrow (BM) cells (50×10⁶); and one or more of CD4⁺ T-depleted spleen cells (25×10⁶), donor CD8⁺ T cells, and donor Granulocyte colony-stimulating factor (G-CSF)-mobilized peripheral blood mononuclear cells from MHC-mismatched C57BL/6 (H-2b) donors.

During HCT, the recipients are co-infused with a population of donor Sox9⁺ cells. Although not required, the population of donor Sox9⁺ cells are preferably derived from the same donor as the HCT cells, and may be delivered with the HCT cells.

Sox9⁺ progenitor cells in the donors may be mobilized into peripheral blood together with marrow hematopoietic progenitor cells into peripheral blood and harvested by apharesis and then transplanted together into the recipient.

After HCT, the recipients are monitored for clinical signs of GVHD and checked for chimerism monthly by staining peripheral blood mononuclear cells with fluorescently labeled anti-donor marker antibody (anti-H-2b). Recipients conditioned with CY, PT, and ATG will generally develop a form of mixed chimerism.

Recipients are treated with growth factors (GE) after HCT and Sox9⁺ cells are delivered. GE is administered as described in the Examples above to induce differentiation of the population of donor Sox9⁺ cells to become insulin-producing 13 cells in vivo. In one embodiment, the donor Sox9⁺ cells may be pre-conditioned with GE prior to infusion, and the GE treatment is then continued in vivo.

As stated above, the foregoing are merely intended to illustrate the various embodiments of the present invention. As such, the specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Castano L & Eisenbarth G S (1990) Type-I diabetes: a chronic autoimmune disease of human, mouse, and rat. Annu Rev Immunol 8:647-679.
2. Fiorina P, Shapiro A M, Ricordi C, & Secchi A (2008) The clinical impact of islet transplantation. Am J Transplant 8(10):1990-1997.
3. Shapiro A M, et al. (2006) International trial of the Edmonton protocol for islet transplantation. The New England journal of medicine 355(13):1318-1330.
4. Wang M, et al. (2012) Mixed Chimerism and Growth Factors Augment beta

Cell Regeneration and Reverse Late-Stage Type 1 Diabetes. Science translational medicine 4(133): 133ra159.
5. Inada A, et al. (2008) Carbonic anhydrase II-positive pancreatic cells are progenitors for both endocrine and exocrine pancreas after birth. Proceedings of the National Academy of Sciences of the United States of America 105(50):19915-19919.
6. Xu X, et al. (2008) Beta cells can be generated from endogenous progenitors in injured adult mouse pancreas. Cell 132(2):197-207.
7. Guz Y, Nasir I, & Teitelman G (2001) Regeneration of pancreatic beta cells from intra-islet precursor cells in an experimental model of diabetes. Endocrinology 142(11):4956-4968.
8. Zulewski H, et al. (2001) Multipotential nestin-positive stem cells isolated from adult pancreatic islets differentiate ex vivo into pancreatic endocrine, exocrine, and hepatic phenotypes. Diabetes 50(3):521-533.
9. Chung C H, Hao E, Piran R, Keinan E, & Levine F (2010) Pancreatic beta-cell neogenesis by direct conversion from mature alpha-cells. Stem cells 28(9):1630-1638.
10. Thorel F, et al. (2010) Conversion of adult pancreatic alpha-cells to beta-cells after extreme beta-cell loss. Nature 464(7292):1149-1154.
11. Zhou Q, Brown J, Kanarek A, Rajagopal J, & Melton D A (2008) In vivo reprogramming of adult pancreatic exocrine cells to beta-cells. Nature 455(7213):627-632.
12. Pan F C, et al. (2013) Spatiotemporal patterns of multipotentiality in Ptf1a-expressing cells during pancreas organogenesis and injury-induced facultative restoration. Development 140(4):751-764.
13. Li W, et al. (2014) Long-term persistence and development of induced pancreatic beta cells generated by lineage conversion of acinar cells. Nature biotechnology 32(12):1223-1230.
14. Solar M, et al. (2009) Pancreatic exocrine duct cells give rise to insulin-producing beta cells during embryogenesis but not after birth. Developmental cell 17(6):849-860.
15. Kopinke D & Murtaugh L C (2010) Exocrine-to-endocrine differentiation is detectable only prior to birth in the uninjured mouse pancreas. BMC developmental biology 10:38.
16. Furuyama K, et al. (2011) Continuous cell supply from a Sox9-expressing progenitor zone in adult liver, exocrine pancreas and intestine. Nature genetics 43(1):34-41.
17. Kopp J L, et al. (2011) Sox9+ ductal cells are multipotent progenitors throughout development but do not produce new endocrine cells in the normal or injured adult pancreas. Development 138(4):653-665.
18. Kopp J L, et al. (2011) Progenitor cell domains in the developing and adult pancreas. Cell cycle 10(12):1921-1927.
19. Weir G C, Aguayo-Mazzucato C, & Bonner-Weir S (2013) beta-cell dedifferentiation in diabetes is important, but what is it? Islets 5(5):233-237.
20. Weir G C & Bonner-Weir S (2013) Islet beta cell mass in diabetes and how it relates to function, birth, and death. Annals of the New York Academy of Sciences 1281:92-105.
21. Bonner-Weir S, et al. (2000) In vitro cultivation of human islets from expanded ductal tissue. Proceedings of the National Academy of Sciences of the United States of America 97(14):7999-8004.
22. Yamada T, et al. (2015) Reprogramming Mouse Cells With a Pancreatic Duct Phenotype to Insulin-Producing beta-Like Cells. Endocrinology 156(6):2029-2038.
23. Jin L, et al. (2013) Colony-forming cells in the adult mouse pancreas are expandable in Matrigel and form endocrine/acinar colonies in laminin hydrogel. Proceedings of the National Academy of Sciences of the United States of America 110(10): 3907-3912.
24. Jin L, et al. (2014) In vitro multilineage differentiation and self-renewal of single pancreatic colony-forming cells from adult C57BL/6 mice. Stem cells and development 23(8):899-909.
25. Suarez-Pinzon W L, Lakey J R, Brand S J, & Rabinovitch A (2005) Combination therapy with epidermal growth factor and gastrin induces neogenesis of human islet {beta}-cells from pancreatic duct cells and an increase in functional {beta}-cell mass. J Clin Endocrinol Metab 90(6):3401-3409.
26. Suarez-Pinzon W L, Yan Y, Power R, Brand S J, & Rabinovitch A (2005) Combination therapy with epidermal growth factor and gastrin increases beta-cell mass and reverses hyperglycemia in diabetic NOD mice. Diabetes 54(9):2596-2601.
27. Rooman I & Bouwens L (2004) Combined gastrin and epidermal growth factor treatment induces islet regeneration and restores normoglycaemia in C57B16/J mice treated with alloxan. Diabetologia 47(2):259-265.
28. Smukler S R, et al. (2011) The adult mouse and human pancreas contain rare multipotent stem cells that express insulin. Cell stem cell 8(3):281-293.
29. Houbracken I, Mathijs I, & Bouwens L (2012) Lineage tracing of pancreatic stem cells and beta cell regeneration. Methods in molecular biology 933:303-315.
30. Lysy P A, Weir G C, & Bonner-Weir S (2012) Concise review: pancreas regeneration: recent advances and perspectives. Stem cells translational medicine 1(2):150-159.
31. Criscimanna A, et al. (2011) Duct cells contribute to regeneration of endocrine and acinar cells following pancreatic damage in adult mice. Gastroenterology 141(4):1451-1462, 1462 e1451-1456.
32. Font-Burgada J, et al. (2015) Hybrid Periportal Hepatocytes Regenerate the Injured Liver without Giving Rise to Cancer. Cell 162(4):766-779.
33. Dor Y, Brown J, Martinez 01, & Melton D A (2004) Adult pancreatic beta-cells are formed by self-duplication rather than stem-cell differentiation. Nature 429(6987):41-46.
34. Pang K, Mukonoweshuro C, & Wong G G (1994) Beta cells arise from glucose transporter type 2 (Glut2)-expressing epithelial cells of the developing rat pancreas. Proceedings of the National Academy of Sciences of the United States of America 91(20):9559-9563.

What is claimed is:

1. A method for treating type 1 diabetes in a subject, comprising:
reversing autoimmunity in the subject; and
replenishing β cells in the subject.

2. The method of claim 1, wherein autoimmunity is reversed by inducing mixed chimerism in the subject.

3. The method of claim 1, wherein β cells are replenished by administering to the subject:
an effective amount of Sox9⁺ cells; and
an effective amount of gastrin or a gastrin receptor agonist, and epidermal growth factor.

4. The method of claim 3, wherein the Sox9⁺ cells are from a donor.

5. The method of claim 3, wherein gastrin or a gastrin receptor agonist, and epidermal growth factor are administered to the subject for an extended period of time.

6. The method of claim 3, wherein gastrin or a gastrin receptor agonist, and epidermal growth factor are administered to the subject at a low dose.

7. The method claim 3, wherein gastrin or a gastrin receptor agonist, and epidermal growth factor are administered under a medium hyperglycemia condition.

8. The method of claim 7, wherein medium hyperglycemia is indicated by a blood glucose level of 300-450 mg/dL.

9. A method for treating type 1 diabetes in a subject, comprising:
administering to the subject low-doses of cyclophosphamide (CY), pentostatin (PT), and anti-thymocyte globulin (ATG);
transplanting in the subject a therapeutically effective amount of donor bone marrow cells;
administering to the subject an effective amount of Sox9⁺ cells; and
administering to the subject an effective amount of gastrin or a gastrin receptor agonist, and epidermal growth factor.

10. The method of claim 9, wherein two or more steps are carried out simultaneously.

11. A composition for treating type 1 diabetes in a subject comprising:
Sox9⁺ cells; and
bone marrow stem cells.

12. The composition of claim 11, further comprising one or more of cyclophosphamide (CY), pentostatin (PT), anti-thymocyte globulin (ATG), gastrin or a gastrin receptor agonist, and epidermal growth factor.
